(19) United States
(12) Patent Application Publication
(10) Pub. No.: US 2015/0150703 A1
(43) Pub. Date: Jun. 4, 2015

(54) Brace Structure

(71) Applicant: Chan-Huang Chang, Ershui Township (TW)

(72) Inventor: Chan-Huang Chang, Ershui Township (TW)

(21) Appl. No.: 14/093,555

(22) Filed: Dec. 2, 2013

(51) Int. Cl. A61F 5/01

(52) U.S. Cl. CPC A61F 5/01

(57) ABSTRACT

A brace structure is placed or is embedded into a shoe and contains a body. The body includes a flexibly protective piece, a shape of which is formed in a U shape so as to correspond to a profile of an ankle. The flexibly protective piece has a support portion defined between two ends thereof so as to support a sole of a foot. The flexibly protective piece also has two lining portions extending upwardly from two ends of the flexibly protective piece so as to cover the ankle. Each lining portion has at least one protecting layer mounted on an inner surface thereof, the flexibly protective piece further has two tabs extending upwardly from two upper ends of the two lining portions, and each tab has at least one fixing portion for corresponding to at least one hole on the shoe.

### FIELD OF THE INVENTION

The present invent relates to a brace structure which after a body of the brace structure is placed into a shoe, at least one protecting layer of two lining portions contacts with an ankle matingly so as to protect the ankle in walking or exercise.

### BACKGROUND OF THE INVENTION

Conventional ankle brace contains a protective sleeve made of a cloth material so as to cover an ankle of a foot. The flexible sleeve has a protecting pad for corresponding to the ankle so as to lower an impact force in walking or exercise.

However, when the ankle brace is in a small size, it forces the ankle tightly, thus causing poor blood circulation.

The present invention has arisen to mitigate and/or obviate the afore-described disadvantages.

### SUMMARY OF THE INVENTION

The primary object of the present invention is to provide a brace structure which after a body of the brace structure is placed into a shoe, at least one protecting layer of two lining portions contacts with an ankle matingly so as to protect the ankle in walking or exercise.

Another object of the present invention is to provide a brace structure which after the body of the brace structure is placed into the shoe, a reaction force resulting from exercise or walking is reduced.

### BRIEF DESCRIPTION OF THE DRAWINGS

FIG. 1 is a perspective view showing the assembly of a brace structure according to a first embodiment of the present invention.

FIG. 2 is a perspective view showing the exploded components of the brace structure according to the first embodiment of the present invention.

FIG. 3 is a perspective view showing the operation of the brace structure according to the first embodiment of the present invention.

FIG. 4 is a perspective view showing the application of the brace structure according to the first embodiment of the present invention.

FIG. 5 is another perspective view showing the application of the brace structure according to the first embodiment of the present invention.

FIG. 6 is a perspective view showing the assembly of a flexibly protective piece of the brace structure according to the first embodiment of the present invention.

FIG. 7 is a perspective view showing the assembly of another flexibly protective piece of the brace structure according to the first embodiment of the present invention.

FIG. 8 is also another perspective view showing the application of the brace structure according to the first embodiment of the present invention.

FIG. 9 is still another perspective view showing the application of the brace structure according to the first embodiment of the present invention.

FIG. 10 is a perspective view showing the assembly of a brace structure according to a second embodiment of the present invention.

### DETAILED DESCRIPTION OF THE PREFERRED EMBODIMENTS

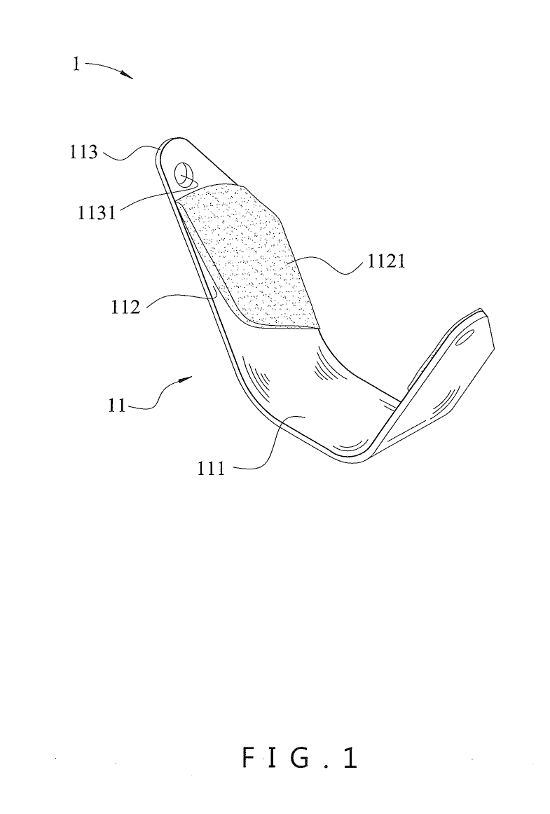

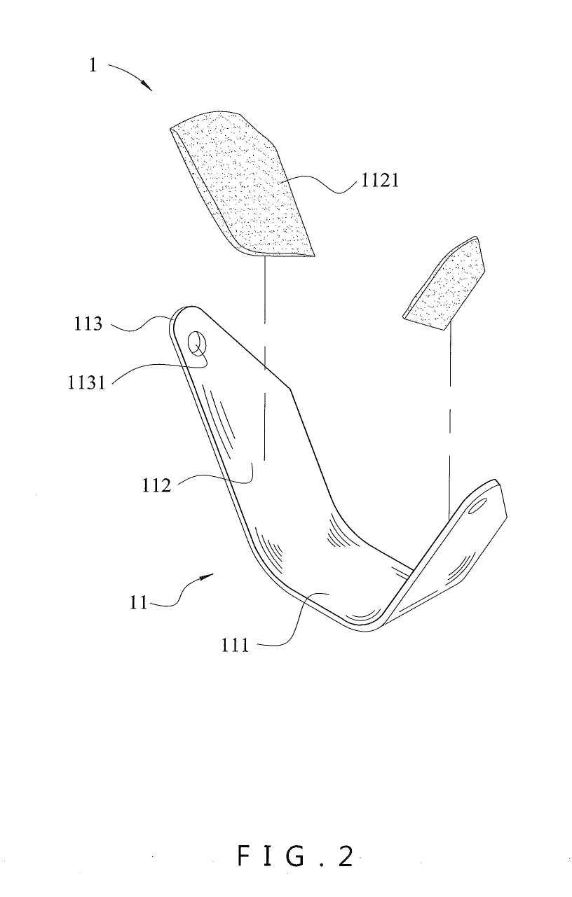

With reference to FIGS. 1-3, a brace structure according to a first embodiment of the present invention comprises a body 1 made of flexible material.

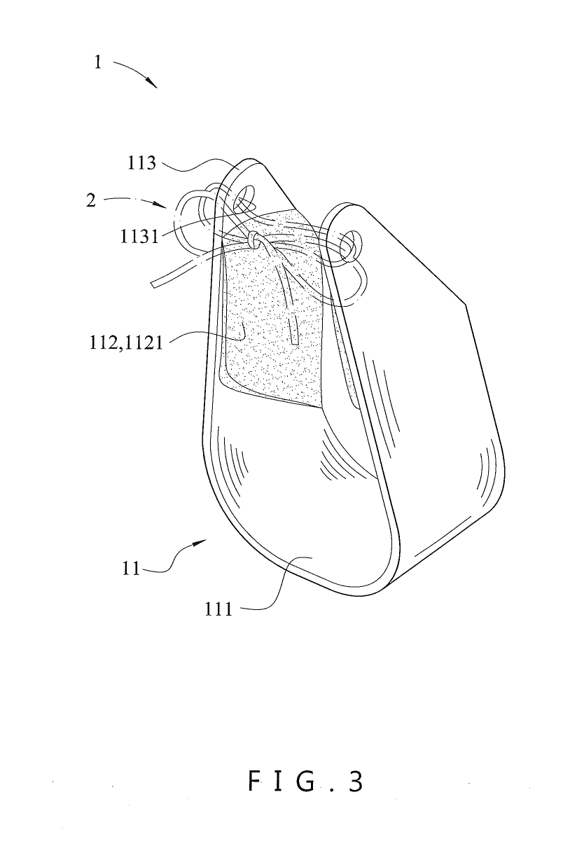

The body 1 includes a flexibly protective piece 11, a shape of which is formed in a U shape so as to correspond to a profile of an ankle. The flexibly protective piece 11 has a support portion 111 defined between two ends thereof and has two lining portions 112 extending upwardly from two ends of the flexibly protective piece 11, wherein each lining portion 112 has at least one protecting layer 1121 mounted on an inner surface thereof. The flexibly protective piece 11 also has two tabs 113 extending upwardly from two upper ends of the two lining portions 112, and each tab 113 has at least one fixing portion 1131 for inserting into a rope 2 (as shown in FIG. 3).

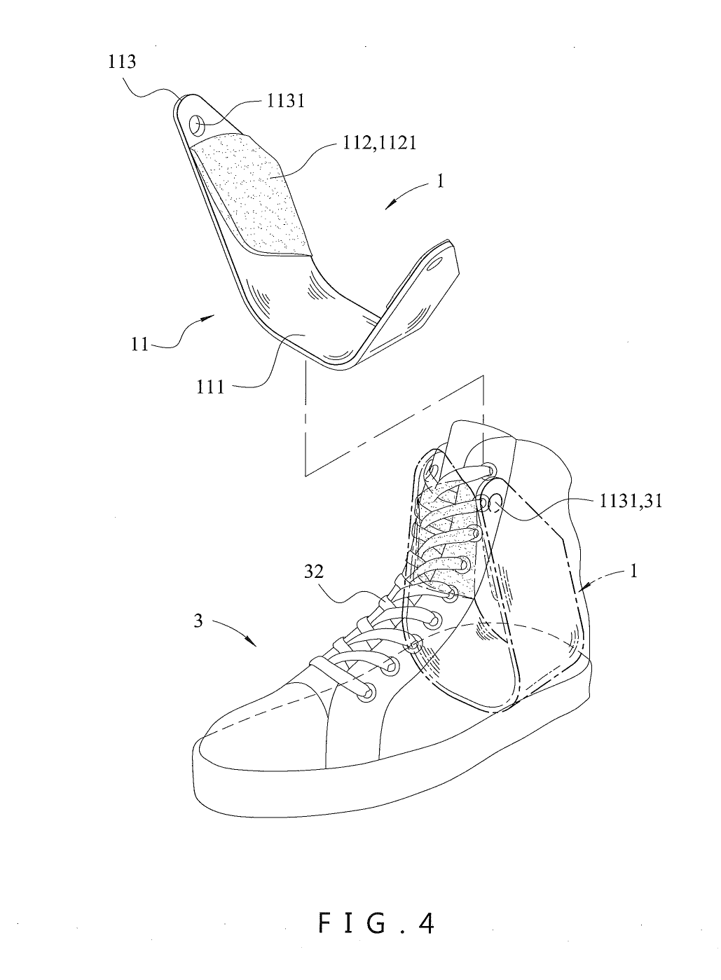

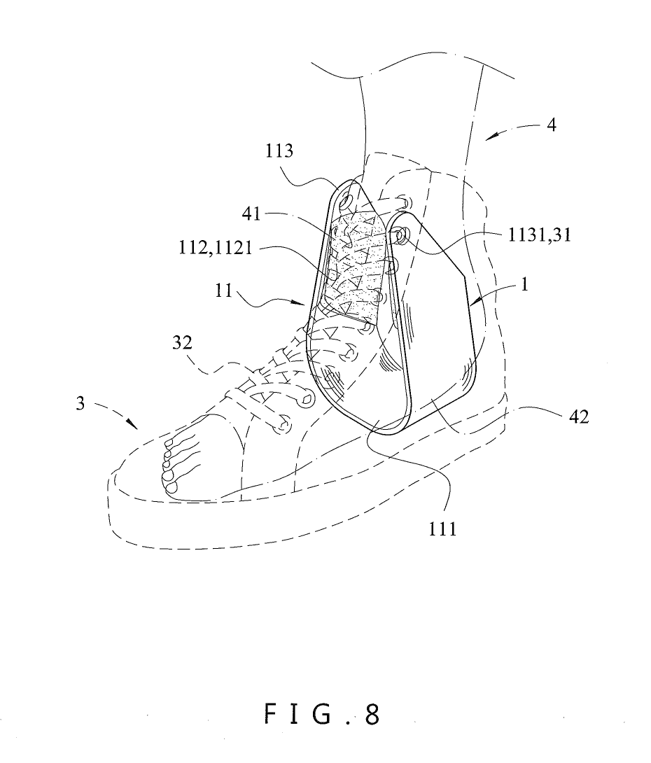

Referring to FIGS. 4-9, the body 1 is used to protect the ankle 41 of a foot 4. The body 1 includes the flexibly protective piece 11, wherein the shape of the flexibly protective piece 11 is formed in the U shape so as to correspond to the profile of the ankle 41, the flexibly protective piece 11 has the support portion 111 defined between the two ends thereof so as to support a sole 42 of the foot 4 and has the two lining portions 112 extending upwardly from the two ends of the flexibly protective piece 11 so as to cover the ankle 41 of the foot 4. Each lining portion 112 has the at least one protecting layer 1121 formed on the inner surface thereof The flexibly protective piece 11 also has the two tabs 113 extending upwardly from the two upper ends of the two lining portions 112, and each tab 113 has the at least one fixing portion 1131 corresponding to at least one hole 31 on a shoe 3.

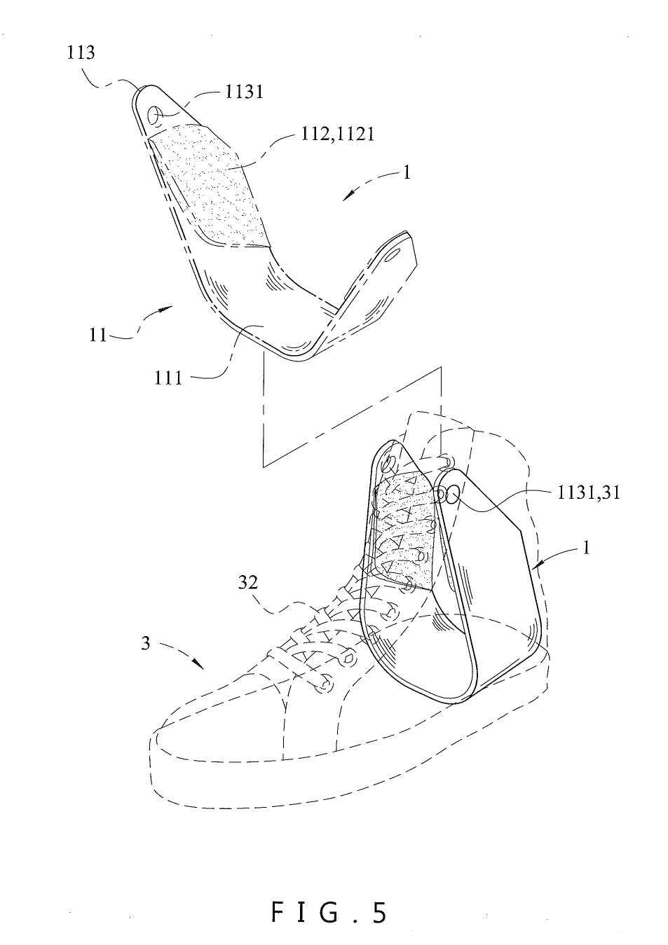

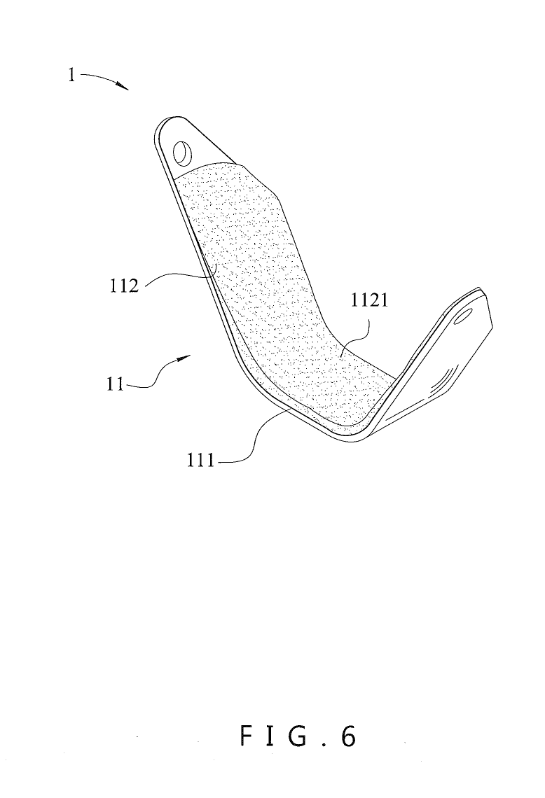

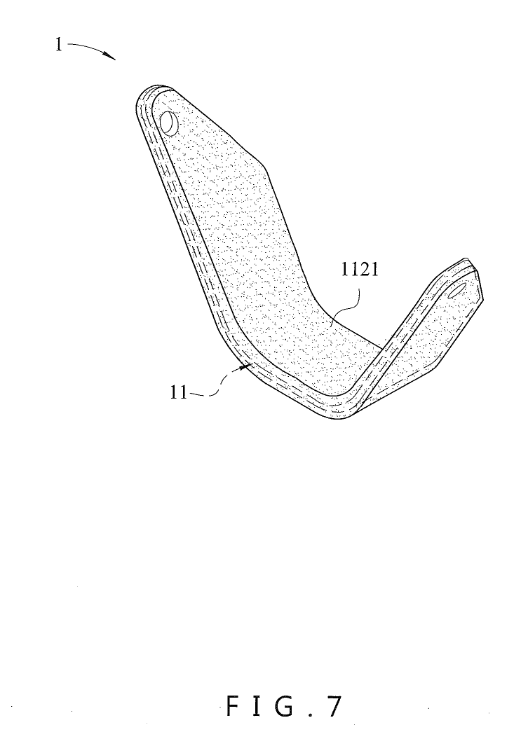

In use, the body 1 is placed into the shoe 3, and a shoelace 32 is inserted through the at least one hole 31 on the shoe 3 and then is tied on the at least one fixing portion 1131 of each tab 113, thus fixing the body 1 in the shoe 3 (as illustrated in FIG. 4). In addition, the body 1 can be embedded into and covered by the shoe 3, and the shoelace 32 is inserted through the at least one hole 31 on the shoe 3 and is tied on the at least one fixing portion 1131 of each tab 113, thus fixing the body 1 in the shoe 3 (as shown in FIG. 5). Preferably, each lining portion 112 has at least one protecting layer 1121 formed on the inner surface thereof so as to correspond to the ankle 41, wherein the at least one protecting layer 1121 is adhered on each lining portion 112 of the flexibly protective piece 11; the at least one protecting layer 1121 is also adhered on the support portion 111 (as shown in FIG. 6). Preferably, the at least one protecting layer 1121 is adhered on a front surface and a back surface of the flexibly protective piece 11 (as shown in FIG. 7) and is made of any one of foam, sponge, rubber and silicone. Thereby, after the body 1 is placed into the shoe 3, the at least one protecting layer 1121 of the two lining portions 112 contacts with the ankle 41 matingly so as to protect the ankle in walking or exercise.

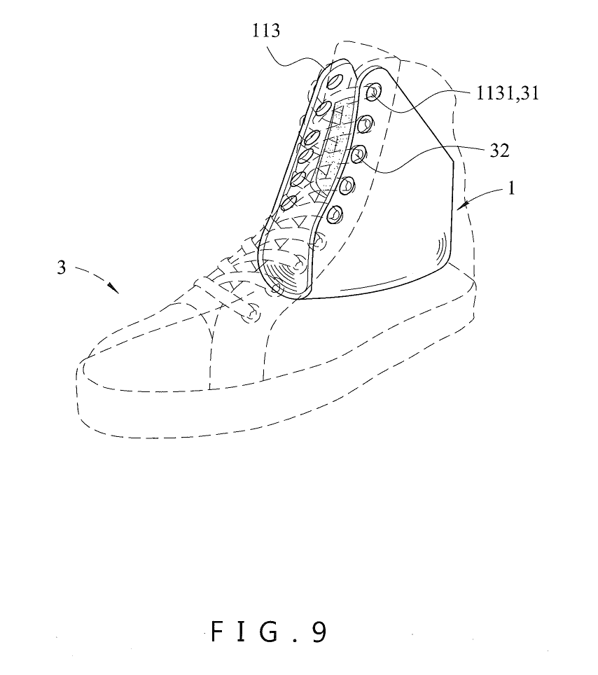

It is to be noted that each tab 113 of the body 1 has the at least one fixing portion 1131 corresponding to the at least one hole 31 on the shoe 3 so that when the body 1 is placed into the shoe 3, the at least one fixing portion 1131 of each tab 113 corresponds to the at least one hole 31 on the shoe 3, hence the shoelace 32 is inserted through the at least one hole 31 on the shoe 3 and is tied on the at least one fixing portion 1131 of each tab 113, thus fixing the body 1 in the shoe 3 (as shown in FIG. 9).

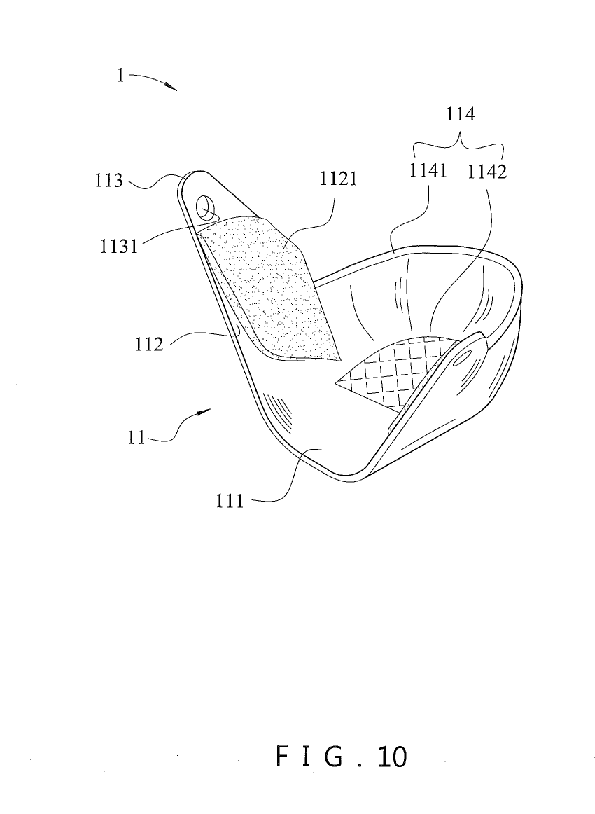

With reference to FIG. 10, a brace structure according to a second embodiment of the present invention comprises a body 1 made of flexible material.

The body 1 includes a flexibly protective piece 11, a shape of the flexibly protective piece 11 is formed in a U shape so as to correspond to a profile of an ankle, the flexibly protective piece 11 has a support portion 111 made of flexible material and defined between two ends thereof and has two lining portions 112 extending upwardly from two ends of the flexibly protective piece 11, wherein each lining portion 112 has at least one protecting layer 1121 mounted on an inner surface thereof. The flexibly protective piece 11 also has two tabs 113 extending upwardly from two upper ends of the two lining portions 112, and each tab 113 has at least one fixing portion 1131; wherein the support portion 111 of the flexibly protective piece 11 further has a heel holding portion 114 extending outwardly from a rear end thereof so as to hold a heel, and the heel holding portion 114 has a surround lining 1141 arranged around a peripheral side of the heel holding portion 114 and connecting with the two lining portions 112 and has a buffer layer 1142 adhered thereon so as to absorb an impact force in walking or exercise. Preferably, the buffer layer 1142 is made of foam material or latex material so as to reduce reaction force in exercise or walking.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

1. A brace structure being placed or embedded into a shoe and comprising a body;
the body including a flexibly protective piece, a shape of which is formed in a U shape so as to correspond to a profile of an ankle, the flexibly protective piece having a support portion defined between two ends thereof so as to support a sole of a foot, the flexibly protective piece also having two lining portions extending upwardly from two ends of the flexibly protective piece so as to cover the ankle, wherein each lining portion has at least one protecting layer mounted on an inner surface thereof, the flexibly protective piece further has two tabs extending upwardly from two upper ends of the two lining portions, and each tab has at least one fixing portion for corresponding to at least one hole on the shoe.

2. The brace structure as claimed in claim 1, wherein the support portion is made of flexible material so that the at least one protecting layer of the two lining portions contacts with the ankle matingly after placing the body in the shoe.

3. The brace structure as claimed in claim 2, wherein the at least one protecting layer is adhered on the support portion and each lining portion of the flexibly protective piece.

4. The brace structure as claimed in claim 2, wherein the at least one protecting layer is adhered on a front surface and a back surface of the flexibly protective piece.

5. The brace structure as claimed in claim 1, wherein the support portion further has a heel holding portion extending outwardly from a rear end thereof so as to hold a heel, and the heel holding portion has a surround lining arranged around a peripheral side of the heel holding portion and connecting with the two lining portions.

6. The brace structure as claimed in claim 5, wherein the heel holding portion has a buffer layer adhered thereon so as to absorb an impact force in walking or exercise.
